(19) United States
(12) Patent Application Publication
(10) Pub. No.: US 2008/0269318 A1
(43) Pub. Date: Oct. 30, 2008

(54) TREATMENT OF AGE-RELATED MACULAR DEGENERATION USING INHIBITORS OF COMPLEMENT FACTOR D

(76) Inventor: Carmelo Romano, Fort Worth, TX (US)

Correspondence Address: ALCON, IP LEGAL, TB4-8, 6201 SOUTH FREEWAY, FORT WORTH, TX, 76134, US

(21) Appl. No.: 12/098,527

(22) Filed: Apr. 7, 2008

Related U.S. Application Data
(60) Provisional application No. 60/914,877, filed on Apr. 30, 2007.

(51) Int. Cl. A61K 31/38; A61P 27/16

(52) U.S. Cl. 514/443

(57) ABSTRACT

The present invention provides methods for identifying a patient at risk for developing AMD by identifying the presence of the Y402H polymorphism or other at risk variants in the complement factor H gene. The present invention further provides methods for treating persons having AMD or at risk for developing AMD as a result of having the Y402H polymorphism or other at risk variants in the complement factor H gene.

The present application claims priority to U.S. Provisional Patent Application 60/914,877 filed Apr. 30, 2007.

### BACKGROUND OF THE INVENTION

1. Field of the Invention

The present invention relates to the field of prevention and treatment of ophthalmic diseases. More specifically, the present invention relates to the prevention and treatment of AMD in patients having at risk variants in complement family genes by administering agents that inhibit complement factor D.

2. Description of the Related Art

Age-related macular degeneration (AMD) is a debilitating, blinding disease that affects the macula or central area of the retina responsible for high-acuity vision and is the leading cause of irreversible vision loss in the elderly. Both genetic and environmental factors are known to play a role in the development of AMD. For example, smoking, lipid intake and age are known risk factors for the development of AMD. The two forms of AMD, dry-AMD and wet-AMD, affect more than 11 million individuals in the US. Dry-AMD occurs in 80% of AMD patients and is characterized by the presence of cellular debris (drusen) in Bruch's membrane under the retinal pigment epithelium (RPE), irregularities in the RPE pigmentation, or geographic atrophy. Wet-AMD, occurring in the remaining 20% of AMD patients, is characterized by choroidal neovascularization and/or detachment of the RPE. Extracellular matrix abnormalities in the eyes of AMD patients have also been implicated.

The diagnosis of dry age-related macular degeneration is defined by the presence of drusen under the RPE and is seen in the early stages of disease. Drusen are small yellowish extracellular deposits composed of protein, lipid, and cellular debris. A major component of drusen are complement proteins [Johnson et al. 2001]. Drusen usually are confluent with significant pigment changes and accumulation of pigment in the posterior pole. RPE often appears atrophic with an easier visualization of the underlying choroidal plexus. In advanced stages of dry AMD, these focal islands of atrophy coalesce and form large zones of atrophy with severely affected vision, a condition referred to as geographic atrophy. Wet AMD is defined by the presence of choroidal neovascularization and may include RPE elevation, exudate, or subretinal fluid.

Vascular endothelial growth factor (VEGF) has been shown to be a key mediator of neovascularization associated with intraocular disorders (Ferrara et al. 1997). The concentration of VEGF in eye fluids are highly correlated to the presence of active proliferation of blood vessels in patients with diabetic and other ischemia-related retinopathies (Aiello et al. 1994). Other studies have demonstrated the localization of VEGF in choroidal neovascular membranes in patients affected by AMD (Lopez et al. 1996). Currently approved treatments for AMD are aimed at reducing excess VEGF, or at blocking its production. For example, the active ingredient in Macugen®, pegaptanib sodium, is a covalent conjugate of an oligonucleotide, which is an antagonist of VEGF. The active ingredient in Lucentis®, ranibizumab, is an antibody fragment that binds VEGF. Macugen® is administered via intravitreal injection every six weeks, whereas Lucentis® is administered via intravitreal injection once a month.

Laser photocoagulation and Photodynamic Therapy are other approved treatments available for wet-AMD. There is currently no treatment to reverse the effects of AMD, however, the Age-Related Eye Disease Study (AREDS) showed that dietary antioxidant supplements may reduce the progression of AMD. [AREDS Report No. 8 (2001)]

A large number of research groups have been intensively searching genes associated with and responsible for the development of AMD. Single nucleotide polymorphism (SNP) genotyping offers great promise in rapidly identifying disease associated genes (Hirschhorn & Daly, 2005; Hinds et al. 2005). Reports published in Science Express and PNAS (Edwards et al. 2005; Haines et al. 2005; Klein et al. 2005; and Hageman et al. 2005) describe the use of SNP genotyping to identify a single polymorphism in the complement factor H gene (CFH) that accounts for elevated risk in developing AMD. A single amino acid change (Y402H) in CFH is reported to account for 40-50% of AMD.

The Edwards study (Edwards et al. 2005) involved scientists at UT Southwestern, Boston University and Sequenom. They performed SNP genotyping through the ARMD1 locus initially using 24 SNPs, then further refining the area with additional SNPs, in two case controlled populations (224 AMD patients and 134 controls in the first population; 176 cases & 68 controls in the second). Edwards et al. report that the individuals with one copy of the Y402H SNP in complement factor H (i.e., heterozygous individuals) had a 2.7× increased risk of developing AMD. This single SNP appears to account for 50% of AMD in their populations.

The Haines study (Haines et al. 2005) was a collaborative study done at Vanderbilt University and Duke University. Similar to the Edwards study, Haines and colleagues SNP genotyped two AMD populations across the ARMD1 locus. Their populations consisted of 182 AMD families with a case control population of 495 AMD patients and 185 controls. Haines et al. initially used 44 SNPs to screen across the ARMD1 locus, then refined their search using additional SNPs. In their overall AMD population they found that heterozygous individuals had a 2.45× elevated risk for AMD, while individuals having both copies of the Y402H SNP (i.e., homozygous individuals) had a 3.33× increased risk for AMD. The risk was even higher for those patients with neovascular (wet) AMD (3.45× in heterozygous individuals and 5.57× in homozygous individuals). Haines et al. estimate that the Y402H SNP is responsible for 43% of AMD in their population.

The Klein study (Klein et al. 2005) involved scientists at Rockefeller University, Yale University, The National Eye Institute (NEI), and EMMES Corporation. Unlike the previous two studies, the Klein group performed a genome-wide SNP genotype screen of 96 AMD patients and 50 controls using >116,000 SNPs. All of the individuals in this study were clinically well-defined from the AREDS study population. The Klein group independently mapped the AMD susceptibility locus to chromosome 1q (the same regions as ARMD 1) and identified the Y402H SNP in CFH as the risk allele. Heterozygous individuals were shown to have a 4.6× elevated risk for AMD, while homozygous individuals had a 7.4× elevated risk for AMD.

The Hageman study (Hageman et al. 2005) included patients from the University of Iowa and Columbia University. Hageman et al. based their analysis of CFH on their previous studies that identified complement in the formation of Drusen and on previous linkage analysis studies that identified the chromosomal locus 1q25-32. The Hageman group analyzed 900 AMD patients and 400 matched controls for SNPs within the CFH gene. In addition to the Y402H variant identified in the previous publications, Hageman et al. identified other AMD risk variants, such as 162V, intervening sequences 1, 2, 6, and 10, A307A, and A473A.

Confirmation of the Edwards, Haines, Klein, and Hageman findings may be found in at least three follow-up studies (i.e., Conley et al. 2005; Zareparsi et al. 2005; and Souied et al. 2005). Conley et al. identified a significant association of the Y402H variant with AMD patients in 796 familial and 196 sporadic AMD cases relative to 120 unaffected, unrelated controls. Zareparsi et al. found that the T>C substitution in exon 9 (Y402H) was associated with AMD in their single center study population. Souied et al. extended the original findings of the Y402H polymorphism association with AMD in the North American populations to the European (French) AMD population. Souied et al. examined 60 sporadic and 81 familial AMD cases and found a significant association of the Y402H polymorphism with AMD relative to 91 healthy controls. Thus, it appears that the Y402H polymorphism association with AMD is a reproducible and generalized finding.

None of the previously described studies propose a treatment regimen for those patients identified as being at risk for developing AMD or for progressing from dry-AMD to wet-AMD due to the presence of the Y402H polymorphism. What is needed is a method for identifying patients at risk for developing AMD and providing a preventative treatment regimen for those patients. Also needed is a treatment regimen for inhibiting vision loss or improving visual acuity in those patients who have already been diagnosed with AMD and are found to possess the Y402H polymorphism or other at risk variants in complement family is genes.

### SUMMARY OF THE INVENTION

The present invention overcomes these and other drawbacks of the prior art by providing a method for treating persons having AMD, or at risk for developing AMD, as a result of having the Y402H polymorphism in the complement factor H(CFH) gene, or other at risk variant in a complement family gene. According to the methods of the invention, a patient is identified as having the Y402H polymorphism, or other at risk variant, in a complement family gene. The identification of the Y402H polymorphism, or other at risk variants, may be accomplished by obtaining tissue, such as by a cheek swab or blood sample, from the patient. The CFH gene, or other complement family gene, is isolated from the tissue by means that are routine for the skilled artisan. The sequence for the gene isolated from the patient is compared with the sequence of the CFH gene, or other complement family gene, not containing the Y402H polymorphism (also referred to as the “normal complement gene” or “wild-type complement gene”) to determine whether the Y402H polymorphism, or other at risk variant, is present in the tissue sample taken from the patient. If the patient is identified as possessing the Y402H polymorphism, or other at risk variant, a composition comprising an inhibitor of complement factor D is administered to the patient to inhibit the loss of visual acuity associated with age-related macular degeneration (AMD) or to prevent the development of AMD in the patient. Thus, the method of the invention comprises the following steps:
a) identifying a Y402H polymorphism, or other at risk variant, in a patient by
i) obtaining a tissue sample from the patient; and
ii) analyzing the tissue sample for the presence of the Y402H polymorphism in the CFH gene, or other at risk variant in a complement family gene, wherein the presence of the Y402H polymorphism in the CFH gene, or other at risk variant in a complement family gene, indicates an increased risk for the development of AMD or for the progression of dry-AMD to wet-AMD;
b) administering to a patient identified in step (a) above as possessing the Y402H polymorphism in the CFH gene, or other at risk variant in a complement family gene, a therapeutically effective amount of a composition comprising an inhibitor of complement factor D.

As used herein, the phrase “complement family gene” refers to any member of the complement pathway, illustrated in FIG. 1. As used herein, the phrase “at risk variant” refers to a difference in the sequence of a gene isolated from a patient as compared to the sequence of the wild-type gene, where such difference has been identified as being linked to an increased incidence of AMD in a particular population of patients.

In another embodiment, the invention provides a method for treating AMD in a patient having been diagnosed with AMD, by administering to the patient a therapeutically effective amount of a composition comprising an inhibitor of complement factor D.

A non-comprehensive list of complement factor D inhibitors within the scope of the present invention include small molecules that inhibit the serine protease activity of the enzyme; inhibitory antibodies; non-antibody proteins that bind to and inactivate factor D; and agents that inhibit the expression of factor D such as small interfering RNAs (siRNA), short hairpin RNAs (shRNA), ribozymes, deoxyribozymes, and antisense RNAs. The amount of complement factor D inhibitor present in the composition of the invention will typically be from 0.01% to 10% percent by weight. In a preferred aspect of the method of the invention, the complement factor D inhibitor is BCX-1470 (see structure below) (Szalai et al. 2000).

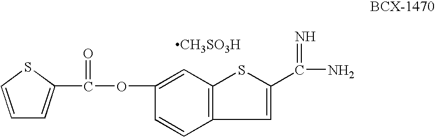

While the compositions of the invention may be delivered by any known means of local ocular delivery, the preferred methods of administration of the composition will be by topical ocular delivery, posterior juxtascleral administration, intravitreal injection, subTenons administration, or by implant, either intravitreal or transscleral. Preferably, the composition of the invention will be administered by posterior juxtascleral administration or by sustained delivery device implanted intravitreally.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1 provides an overview of the complement system, illustrating the classical, MB-Lectin, and alternative pathways.

### DETAILED DESCRIPTION PREFERRED EMBODIMENTS

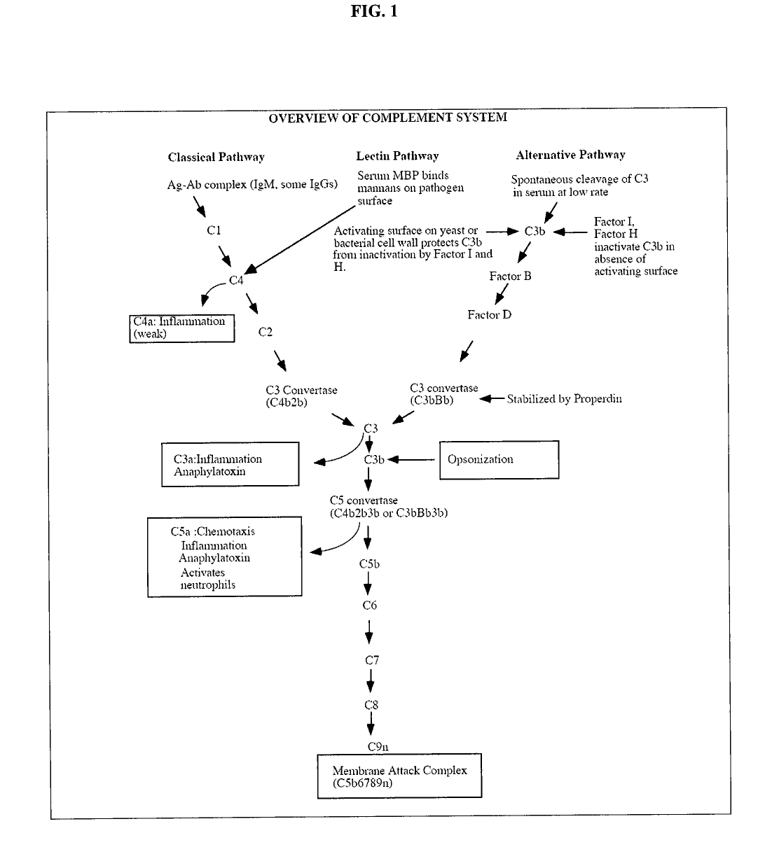

It has recently been reported that a single nucleotide polymorphism (SNP) in complement factor H(CFH) is responsible for nearly 50% of the attributable risk of AMD (Edwards et al., 2005; Haines et al. 2005; Klein et al. 2005; Hageman et al. 2005). The normal function of CFH appears to be to prevent excess complement activation. The complement system complements and amplifies the body's antibody response to foreign pathogens and is composed of three pathways: classical, MB-lectin, and alternative (FIG. 1).

In the alternative complement pathway, the thioester bond of a small percentage of the plasma-resident, biologically inert protein C3 is spontaneously hydrolyzed to form C3(H₂O). This hydrolyzed C3 has a much higher binding affinity than C3 itself for the plasma protein factor B, with which it forms a non-covalent complex. The C3(H₂O)-factor B complex is a substrate for the plasma serine protease factor D, which cleaves factor B into two new proteins, the small fragment Ba and the active protease Bb, the latter remaining associated with C3(H₂O) to form the C3(H₂O)Bb complex. This complex is a fluid-phase C3 convertase, and it can cleave many molecules of C3 to C3a, a pro-inflammatory chemoattractant for leukocytes like neutrophils, and C3b, an opsonization agent that labels cells for ingestion by professional phagocytes. Much of the so-formed C3b is inactivated by hydrolysis, but some attaches covalently, through its reactive thioester group, to the surfaces of host cells or to pathogens. Cell surface-deposited C3b is able to bind factor B, allowing its cleavage by factor D to yield the small fragment Ba and the active protease Bb. This results in formation of the alternative pathway C3 convertase, C3bBb, on cell surfaces. The cell surface-bound C3bBb C3 convertase can bind another molecule of C3b to form the C5 convertase C3bBbC3b. This C5 convertase reacts with C5 to release the potent chemoattractant C5a into plasma. The residual C5-derived fragment C5b recruits the proteins C6-C9 to form the membrane attack complex (MAC), an oligomeric protein complex that causes lysis by forming a pore in the plasma membrane of the cell to which it is attached.

Inhibition of factor D function by a variety of means, such as inhibition of its expression, binding by an anti-factor D antibody or aptamer, or inhibition of its serine protease activity, could in theory represent a strategy for reducing activation of the alternative complement system, by reducing formation of the opsonization agent C3b and thus reducing phagocytosis of the labeled cell, and by reducing formation of the MAC, thus reducing cell lysis. This may reduce the contribution of inappropriate alternative complement system activation to AMD pathology/tissue destruction. Inhibition of complement factor D as a means of preventing and/or ameliorating AMD-associated disease pathology in man has not been attempted.

The present invention relates to the prevention and treatment of AMD by inhibiting complement factor D. The target patient population of complement factor D inhibitor therapy may be identified by genetic screening, e.g. using a cheek swab or blood analysis, and genotyping for the Y402H SNP or other at risk variants. Genomic DNA may be isolated from peripheral blood leukocytes using QIAamp DNA Blood Maxi Kits (Qiagen, Valencia, Calif.). DNA polymorphisms may be detected by single-strand conformational polymorphism (SSCP) analyses, using Applied Biosystems SNP Assays-On-Demand quantitative PCR, or by direct sequencing of amplified DNA. Other means of detecting polymorphisms in the CFH gene are included and will be routine to the skilled artisan.

The complement factor D inhibitors of the present invention can be administered either systemically or locally. Systemic administration includes: oral, transdermal, subdermal, intraperitioneal, subcutaneous, transnasal, sublingual, or rectal. The most preferred systemic route of administration is oral. Local administration for ocular administration includes: topical, intravitreal, periocular, transcleral, retrobulbar, juxtascleral, sub-tenon, or via an intraocular device. Preferred methods for local delivery include transscleral delivery to the macula by posterior juxtascleral administration; via intravitreal injection; or via cannula, such as that described in U.S. Pat. No. 6,413,245b1. Alternatively, the inhibitors may be delivered via a sustained delivery device implanted intravitreally or transsclerally, or by other known means of local ocular delivery.

The present invention is also directed to compositions containing complement factor D inhibitors and analogs, and methods for their use. According to the methods of the present invention, a composition comprising one or more compounds of the present invention and a pharmaceutically acceptable carrier for systemic or local ocular administration is administered to a mammal in need thereof. Preferred compositions for use in the methods of the present invention contain a complement factor D inhibitor, such as BCX-1470. The compositions are formulated in accordance with methods known in the art for the particular route of administration desired.

According to the methods of the present invention, a composition comprising one or more complement factor D inhibitors and a pharmaceutically acceptable carrier for systemic or local administration is administered to a mammal in need thereof.

The compositions administered according to the present invention comprise a pharmaceutically effective amount, or therapeutically effective amount, of one or more complement factor D inhibitors. As used herein, a “pharmaceutically effective amount” or “therapeutically effective amount” is an amount of active agent that is sufficient to reduce or prevent AMD and/or the loss of visual acuity associated with AMD. Generally, for compositions intended to be administered systemically for the treatment of AMD, the total amount of complement factor D inhibitor will be about 0.01-100 mg/kg. For local administration, the preferred concentration of complement factor D inhibitor in the composition will be from about 0.01% to about 10% [w/v].

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

| Ingredients | Amount (wt %) |
|---|---|
| BCX-1470 | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 2

| Ingredients | Amount (wt %) |
|---|---|
| BCX-1470 | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 3

| Ingredients | Amount (wt %) |
|---|---|
| BCX-1470 | 0.01-2% |
| Guar Gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 4

| Ingredients | Amount (wt %) |
|---|---|
| BCX-1470 | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

### Example 5

10 mM IV Solution w/v %

| | |
|---|---|
| BCX-1470 | 0.384% |
| L-Tartaric acid | 2.31% |
| Sodium hydroxide | pH 3.8 |
| Hydrochloric acid | pH 3.8 |
| Purified water | q.s. to 100% |

### Example 6

5 mg Capsules

| | mg/capsule |
|---|---|
| Ingredient | (Total Wt. 100 mg) |
| BCX-1470 | 5 |
| Lactose, anhydrous | 55.7 |
| Starch, Sodium carboxy-methyl | 8 |
| Cellulose, microcrystalline | 30 |
| Colloidal silicon dioxide | .5 |
| Magnesium stearate | .8 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents and Published Applications
U.S. Pat. No. 6,413,245b1
2003/0207309

Books

Other Publications
AREDS Report No. 8, “A Randomized, Placebo-Controlled, Clinical Trial of High-Dose Supplementation with Vitamins C and E, Beta Carotene, and Zinc for Age-Related Macular Degeneration and Vision Loss,” Arch Opthalmol, (2001), pp. 1417-1436, Vol. 119, JAMA & Archives
Edwards, A. O., Ritter, R., 3rd, Abel, K. J., Manning, A., Panhuysen, C., Farrer, L. A., Haines, J. L., Hauser, M. A., Schmidt, S., Scott, W. K., et al. (2005). Complement factor H polymorphism and age-related macular degeneration. Science 308, 421-424.
Furlong, S. T., Dutta, A. S., Coath, M. M., Gormley, J. J., Hubbs, S. J., Lloyd, D., Mauger, R. C., Strimpler, A. M., Sylvester, M. A., Scott, C. W., and Edwards, P. D. (2000). C3 activation is inhibited by analogs of compstatin but not by serine protease inhibitors or peptidyl alpha-ketoheterocycles. Immunopharmacology 48, 199-212.
Hageman, G. S., Anderson, D. H., Johnson, L. V., Hancox, L. S., Taiber, A. J., Hardisty, L. I., Hageman, J. L., Stockman, H. A., Borchardt, J. D., Gehrs, K. M., et al. (2005). A common haplotype in the complement regulatory gene factor H(HF1/CFH) predisposes individuals to age-related macular degeneration. Proc Natl Acad Sci USA 102, 7227-7232.
Haines, J. L., Hauser, M. A., Schmidt, S., Scott, W. K., Olson, L. M., Gallins, P., Spencer, K. L., Kwan, S. Y., Noureddine, M., Gilbert, J. R., et al. (2005). Complement factor H variant increases the risk of age-related macular degeneration. Science 308, 419-421.
Johnson, L. V.; Leitner, W. P.; Staples, M. K.; Anderson, D. H. (2001). Complement activation and inflammatory processes in drusen formation and age related macular degeneration. Experimental Eye Research 73(6), 887-896.
Klein, R. J., Zeiss, C., Chew, E. Y., Tsai, J. Y., Sackler, R. S., Haynes, C., Henning, A. K., SanGiovanni, J. P., Mane, S. M., Mayne, S. T., et al. (2005). Complement factor H polymorphism in age-related macular degeneration. Science 308, 385-389.
Ferrara et al. Endocr. Rev. 18:4-25 (1997).
Aiello et al. N. Engl. J. Med. 331:1480-1487 (1994).
Lopez et al. Invest. Ophtalmo. Vis. Sci. 37:855-868 (1996).
Szalai, A. J.; Digemess, S. B.; Agrawal, A.; Kearney, J. F.; Bucy, R. P.; Niwas, S.; Kilpatrick, J. M.; Babu, Y. S.; Volanakis, J. E. (2000) The Arthus reaction in rodents: species-specific requirement of complement. Journal of Immunology 164(1), 463-468.

We claim:

1. A method for inhibiting the loss of visual acuity associated with age-related macular degeneration (AMD) in a patient having AMD or at risk for developing AMD due to the presence of an at risk variant in a complement family gene, said method comprising:
a) identifying said at risk variant in said patient by
i) obtaining a tissue sample from said patient; and
ii) assaying said tissue sample for the presence of the at risk variant, wherein the presence of the at risk variant indicates an increased risk for the development of AMD or for the progression of dry-AMD to wet-AMD;
b) administering to a patient identified in step (a) above as possessing the at risk variant a therapeutically effective amount of a composition comprising a complement factor D inhibitor.

2. The method of claim 1, wherein the complement factor D inhibitor is BCX-1470.

3. The method of claim 1, wherein the amount of complement factor D inhibitor in the composition is from 0.01 to 10 percent by weight.

4. The method of claim 1, wherein the at risk variant is a Y402H polymorphism in the complement factor H gene.

5. The method of claim 1, wherein the composition is administered via a method selected from the group consisting of oral administration, topical ocular administration, intravitreal injection, periocular administration, juxtascleral administration, retrobulbar administration, sub-tenon administration, transscleral, and via an intraocular device.

6. The method of claim 5, wherein the composition is administered by posterior juxtascleral administration.

7. The method of claim 6, wherein the composition is administered by sustained delivery device implanted intravitreally.

8. An ophthalmic composition comprising a complement factor D inhibitor in a pharmaceutically acceptable carrier.

9. The composition of claim 8, wherein the complement factor D inhibitor is BCX-1470.

10. The composition of claim 8, wherein the concentration of complement factor D inhibitor in the composition is from about 0.01% to about 2% (w/v).
